(19) United States
(12) Patent Application Publication
(10) Pub. No.: US 2011/0166149 A1
(43) Pub. Date: Jul. 7, 2011

(54) PTERIDINE DERIVATIVES FOR TREATING RESPIRATORY DISEASE

(75) Inventors: Luis A. Dellamary, San Marcos, CA (US); Michael B. Martin, San Marcos, CA (US)

(73) Assignee: TARGEGEN INC., San Diego, CA (US)

(21) Appl. No.: 12/866,665

(22) PCT Filed: Feb. 6, 2009
(86) PCT No.: PCT/US2009/033353

§ 371 Date: Feb. 16, 2011

Related U.S. Application Data
(60) Provisional application No. 61/027,180, filed on Feb. 8, 2008.

(51) Int. Cl. A61K 31/519; A61P 11/00; A61P 11/08; A61P 11/06

(52) U.S. Cl. 514/249

(57) ABSTRACT

The invention provides methods and compositions for treating asthma and/or COPD. For example, provided herein are compositions that include a kinase inhibiting agent such as 6,7-bis(3-hydroxyphenyl)-pteridine-2,4-diamine or pharmaceutically acceptable salts thereof; and a surfactant.

### CROSS-REFERENCE TO RELATED APPLICATION

This application claims the benefit of priority under 35 U.S.C. §119(e) to U.S. Provisional Application No. 61/027,180, filed Feb. 8, 2008, the entire contents of which are incorporated herein by reference.

### BACKGROUND

Airway diseases, such as asthma and chronic obstructive pulmonary disease (COPD), have been increasing in prevalence in recent years. The Centers for Disease Control and Prevention has estimated that 17 million American adults have been diagnosed with asthma and another 10 million American adults have been diagnosed with COPD.

Asthma is a long-term lung disease characterized in part by inflammation of the lower airways and episodes of airflow obstruction. Asthma severity ranges from intermittent mild symptoms, such as coughs and wheezing, to severe, life-threatening attacks that require immediate hospital treatment. Obstruction of the airway in asthma is generally considered reversible, meaning that the obstruction of the lung can generally be resolved with treatment and in some cases can resolve spontaneously.

COPD refers to a group of diseases that cause airflow blockage and breathing-related problems. These diseases include emphysema, chronic bronchitis, and, in some cases, asthma. COPD is a progressive condition in which the airways narrow and become obstructed, making it difficult to breathe, eventually leading to a long-term disabling breathlessness.

Local administration of pharmaceutically effective agents for use, e.g. with asthma and/or COPD can be a more effective and safer treatment compared to oral or injectable agents in part because much higher concentrations of the drug can be delivered to the diseased tissue without exposing the rest of the healthy tissue. However, topical/local administration of agents may eventually drain into the GI tract. If the agent is orally bioavailable, systemic concentrations may occur that can render the treatment unsafe or cause some side effects, specially with highly potent drugs (with IC₅₀ of less than 500 nM).

There is a on-going need for improved formulations and agents that are effective for asthma and/or COPD, and that may have minimal side effects for use in the treatment of asthma and/or COPD.

### SUMMARY

The present invention provides a method of treating asthma and/or COPD, for example emphysema and/or chronic bronchitis. A method of treating or ameliorating asthma and/COPD in a patient in need thereof is disclosed, comprising administering to the patient a composition suitable for inhalation or nasal administration, wherein the composition comprises a kinase inhibiting agent, for example, a compound of structure (I), as disclosed herein.

For example, disclosed herein are methods for targeting delivery of a pharmaceutical kinase inhibitor composition to the respiratory tract of a patient in need thereof, comprising administering to the patient a therapeutically effective amount of pharmaceutically acceptable composition suitable for direct delivery to the respiratory tract, where such composition may comprise from about 0.00001 to about 10% w/v of an agent selected from: 6,7-bis(3-hydroxyphenyl)-pteridine-2,4-diamine or pharmaceutically acceptable salts thereof; and from about 0.00001 to about 10% w/v of a surfactant, wherein the administration results in the drug being predominantly delivered to a mucosal surface of the respiratory tract of the patient and results in a minimal plasma concentration of the kinase inhibiting agent in the patient. In some embodiments, a disclosed pharmaceutically acceptable composition further comprises an aqueous based solvent.

In some embodiments, the methods may result in a plasma concentration of the agent in the patient is less than about 10 ng/mL, or less than about 2 ng/mL within about two hours after administration.

Exemplary disclosed compositions include an agent represented by Formula I, below, for example, 6,7-bis(3-hydroxyphenyl)-pteridine-2,4-diamine or pharmaceutically acceptable salts thereof, and a surfactant. For example, a contemplated composition may include about 0.00001% w/v to about 20% w/v of 6,7-bis(3-hydroxyphenyl)-pteridine-2,4-diamine or pharmaceutically acceptable salts thereof; about 0.00001% w/v to about 6% w/v of a surfactant; and water sufficient to make 100% w/v.

Exemplary surfactants for use in contemplated compositions may be, for example, selected from one or more of: tyloxapol, poloxamer 188, poloxamer 407, tween 80, phosphatidylcholine, phasphatides, and phophatidyl glycerols. In an embodiment, the surfactant is tyloxapol. Disclosed compositions may be in any form, e.g. in the form of a nebulized aerosol or a powder.

When administered to a patient as a nebulized aerosol or dry powder, the composition may result in a lung tissue concentration in the patient of at least about 10 ng/g of 6,7-bis(3-hydroxyphenyl)-pteridine-2,4-diamine or pharmaceutically acceptable salts thereof about 15 minutes after administration.

Also disclosed herein is a kit for treating a respiratory disease, the kit comprising (a) a composition comprising 6,7-bis(3-hydroxyphenyl)-pteridine-2,4-diamine or pharmaceutically acceptable salts thereof, and a non-ionic surfactant, in a sealed container, and (b) a label indicating administration by inhalation or nasally. Such kits may include a composition comprising 0.05 mg to 40 mg of 6,7-bis(3-hydroxyphenyl)-pteridine-2,4-diamine or pharmaceutically acceptable salts thereof.

### BRIEF DESCRIPTION OF THE DRAWINGS

FIG. 1 shows the concentration time course of compound A in mouse lung tissues following lung exposure.

FIG. 2 shows the concentration time course of compound A in BALF (lung fluid) following lung exposure.

FIG. 3 depicts A) estimated in vivo/in-vitro lung exposure graph; B) depicts the particle size distribution, as effective cut diameter of a disclosed aerosolized composition.

FIG. 4 depicts results of a methacholine challenge experiment in ovalbumin challenged mice.

FIG. 5 depicts the particle size distribution (effective cut diameter) of an aerosolized dry powder that includes compound A: dimyristoylphosphatydyl choline (DMPC): m-Hetastarch: tyloxapol 4:4:4:1 w/w/w/w.

### DETAILED DESCRIPTION

This disclosure is, in part, directed to compositions suitable for nasal administration or by inhalation, and includes compositions comprising: an active agent represented formula I:

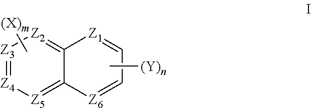

wherein:
each of Z₂ and Z₄ is C, each of Zᵢ, Z₃, Z₅, and Z₆ is N;
each X is NH₂;
each Y is independently selected from a group consisting of —OR^{d}, —NR^{d}₂, —SR^{d}, and —OPO₃H₂, wherein Rd is selected from a group consisting of H, lower alkyl, aryl, and —(CH₂)₂NH(CH₂CH₃); or
each Y is independently selected from a group consisting of alkyl, substituted alkyl, aryl, substituted aryl, and halogen, wherein said substituents are selected from a group consisting of halogen, —OR^{e}, —NR₂—SR^{e}, and —P(O)(OH)₂, wherein R^{e} is selected from a group consisting of —H, lower alkyl, and aryl; or
each Y is independently selected from a group consisting of CH₂glycinyl, CH₂NHethoxy, CH₂NHCH₂alkyl, CH₂NHCH₂t-Bu, CH₂NHCH₂aryl, and CH₂NHCH₂substituted aryl; or
when n is 2, each Y is taken together to form a fused aromatic ring system; and
m and n are each independently 1 to 4, or pharmaceutically acceptable salts thereof; and a pharmaceutically acceptable excipient or surfactant.

An exemplary composition includes the active agent 6,7-bis(3-hydroxyphenyl)-pteridine-2,4-diamine or pharmaceutically acceptable salts there of, as shown below in formula A, and referred to herein as compound A. Compound A, is a yellow powder with high water solubility at pH below 2 and above 10 (solubility greater than 20 mg/mL), with negligible solubility between pH of 3 and 9. It readily dissolves in DMSO and selected aprotic high solvents with high dielectric constants.

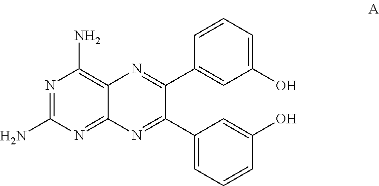

For example, contemplated compositions can include about 0.00001% w/v to about 20% w/v of a compound of formula I, e.g., 6,7-bis(3-hydroxyphenyl)-pteridine-2,4-diamine or pharmaceutically acceptable salts thereof. The compositions may include about 0.001% to about 5%, to about 10%, or even to about 25% of such compounds, or may include about 0.00001% w/v to about 0.01% or to about 0.01%, or even to about 0.001%.

Such compositions may also include a surfactant, e.g. a non-ionic surfactant, for example, about 0.0001% w/v to about 6% w/v of a surfactant, e.g. a non-ionic surfactant, or about 0.00001% w/v to about 10%, to about 5%, or even to about 4% w/v. In some embodiments, compositions disclosed herein may include an aqueous based solvent, e.g. water.

Contemplated surfactants include tyloxapol, poloxamer 188, poloxamer 407, tween 80, phosphatidylcholine (e.g. pure or mixtures of chain lengths of C₁₀ to C₂₀, saturated or unsaturated), phosphatides, and phophatidyl glycerols, or combinations of different surfactants. In an embodiment, the surfactant is tyloxapol.

Dry powder compositions can include about 0.1% to about 90% w/w of a compound of formula I, for example, about 0.1% to about 10%, 20% or 50% w/w. Such dry powder compositions may also include surfactants, e.g., tyloxapol or phospholipids, and/or may include other excipients e.g. starches, lactose, mannitol, sugar, amino acids, chloride salts of sodium, potassium and/or calcium.

Compositions can be in the form of a nebulized aerosol or a powder; or can be part of a liquid, a solution, an aqueous suspension, a non-aqueous suspension (e.g. as part of a formulation that includes hydrofluoroalkanes, e.g. HFA 134a or HFA 227ea, chlorofluorocarbons, hydrochlorofluorocarbons, perfluorocarbons or combinations thereof. Such formulations may include ethyl alcohol up to about 50% v/v, e.g., about 10% v/v to about 40% v/v. Compositions may be included in a solid formulation, e.g., compositions may include polyols, and/or carbohydrates (sugars), and/or aminoacids, or a mixture of solids (e.g. including carriers or dispersants), and can be used as is or mixed with another suitable pharmaceutical excipient before use. Such compositions are suitable for delivery via inhalation or administered via or to the nose.

The methods and composition disclosed herein contemplate compositions that include compounds of Formula I administered alone or in combination with other agents (e.g., protein therapeutic agents or short-acting agents, for example albuterol). For example, contemplated compositions may contain other therapeutic agents, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques known in the art of pharmaceutical formulation.

The compounds disclosed herein may be formulated into therapeutic compositions as natural or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts include amine salts formed by the protonation of an amino group with inorganic acids such as hydrochloric acid, hydrobromic acid, hydroiodic acid, sulfuric acid, phosphoric acid, and the like. Salts of the invention also include amine salts formed by the protonation of an amino group with suitable organic acids, such as p-toluenesulfonic acid, acetic acid, and the like. In addition, polymorphs of compounds are contemplated herein.

Disclosed compounds and compositions may be administered via inhalation or nasally, e.g. by a single method or combination of 1) dry powder inhalation (passive or active), 2) pressurized metered dose inhalers, 3) instillation, 4) nebulization (jet or ultrasonic), 5) soft mist inhalers. In an embodiment, this disclosure contemplated a nebulized aerosol of a dispersion of droplets, e.g. liquid droplets, that have a particle size of about 100 nm to about 20 microns in diameter and/or comprise a liquid, a crystalline agent of formula I, and a surfactant, e.g. a non-ionic surfactant.

In another embodiment, the disclosed agents, e.g. compounds of formula I, can be made into a form or composition suitable for use in a nasal or inhaled formulation, e.g. a dry powder formulation, that can be achieved by, for example, 1) spray drying, 2) wet milling 3) micronization 4) supercritical fluid particle formation (e.g., spray gas, MicronMix™, particles from gas saturated solutions, rapid expansion from supercritical solutions, etc). In an embodiment, a dry powder formulation can be suspended in non-aqueous liquids, for example, chlorofluorocarbons, hydrofluoroalkanes, hydrochlorofluorocarbons, and/or fluorocarbons, which may be used in pressurized metered dose inhalers or aerosolized as dry powder in active or passive inhalers.

Disclosed compositions, when administered to a patient nasally or by inhalation, results in a lung tissue concentration in the patient at least about 10 ng/g of the active agent, e.g, 6,7-bis(3-hydroxyphenyl)-pteridine-2,4-diamine or pharmaceutically acceptable salts thereof about 15 minutes, about 20 minutes, or about 30 minutes after administration.

In another embodiment, disclosed compositions, when administered to a patient by inhalation or nasally, e.g. as a nebulized or aerosolized dry powder formulation, results in a plasma concentration in the patient of less than about 5 ng/mL, less than about 3 ng/mL, less than about 100 ng/mL, about 15 minutes, or about 20 minutes after administration, e.g. about 5 to about 30 minutes after administration.

Also contemplated herein is a kit for use in the treatment of a respiratory disease, the kit comprising (a) a suspension or dry powder of a disclosed compound, e.g., 6,7-bis(3-hydroxyphenyl)-pteridine-2,4-diamine or pharmaceutically acceptable salts thereof, and a surfactant, in a sealed container, and (b) a label indicating administration by inhalation or nasally. Such a suspension may contain about 0.05 mg to about 40 mg of e.g, 6,7-bis(3-hydroxyphenyl)-pteridine-2,4-diamine or pharmaceutically acceptable salts thereof.

Although dosage amounts are disclosed herein, it will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of asthma or COPD, and the host undergoing therapy.

In an embodiment, a method of treating asthma or COPD of a patient in need thereof is provided, the method comprising administering by inhalation to the patient a composition comprising a composition disclosed herein, e.g., a therapeutically effective amount of an active agent of formula I, e.g., 6,7-bis(3-hydroxyphenyl)-pteridine-2,4-diamine or pharmaceutically acceptable salts thereof and a surfactant, thereby delivering to the mucus membranes of the respiratory tract of the patient a pharmaceutically effective amount of the active agent. Contemplated methods include treating emphysema and/or chronic bronchitis.

Such methods may result in a plasma concentration of the agent in the patient of less than about 10 ng/mL, or less than about 5 ng/mL, or less than about 2 ng/mL two hours after administration.

The term “therapeutically effective amount” refers to the amount of the compound or pharmaceutical composition that will elicit the biological or medical response of a tissue, system, animal or human that is being sought by the researcher, veterinarian, medical doctor or other clinician, e.g., restoration or maintenance of eyesight and/or amelioration of an retinal vein occlusion.

The term “pharmaceutically acceptable” refers to the fact that the carrier, diluent or excipient must be compatible with the other ingredients of the formulation and not deleterious to the recipient thereof.

The terms “administration of a compound” or “administering a compound” refer to the act of providing a compound of the invention or pharmaceutical composition to the subject in need of treatment.

Also disclosed herein is a method for targeting delivery of a pharmaceutical kinase inhibitor composition to the respiratory tract, e.g. to the mucosal surface of the respiratory tract, of a patient in need thereof, comprising administering to the patient a therapeutically effective amount of pharmaceutically acceptable composition suitable for direct delivery to the respiratory tract such as disclosed herein, e.g a composition comprising (a) from about 0.00001 to about 10% w/v of an agent of formula I, e.g., selected from: 6,7-bis(3-hydroxyphenyl)-pteridine-2,4-diamine or pharmaceutically acceptable salts thereof; and (b) from about 0.00001 to about 10% w/v of a surfactant, wherein the administration results in the drug being predominantly delivered to the mucosal surface of the patient and results in a minimal plasma concentration of the kinase inhibiting agent in the patient.

### EXAMPLES

The following examples are provided to further illustrate the advantages and features of the present invention, but are not intended to limit the scope of the invention.

### Example 1

### Lung Exposure and Particle Size Analysis

6,7-bis(3-hydroxyphenyl)-pteridine-2,4-diamine (Compound A) was formulated as a 20% suspension in a 5% tyloxapol solution in water. Particle size reduction was achieved using a colloid mill (magicLAB®, MK module, for 6 hours @ 12,000 rpm, while maintaining a temperature below 50° C.). Samples at 4, 0.4, 0.04 and 0.004% of compound A were prepared by dilution with DI water.

Nebulizations of the suspensions were carried out using a Pari LC Plus @ 3 LPM, using an active diluter at 5 LPM in a CH technologies nose-only exposure chamber. Aerodynamic particle size was determined by a 7 stage impactor from Intox at 1 LPM. Each stage extracted with acetonitrile:water 1:1 with 0.05% TFA and analyzed by HPLC against an external standard.

Estimated lung exposure levels were calculated based on the following assumptions: respirable dose collected from 7 stage impactor; 30 minute exposure time; mouse minute volume of 23 mL/min; mouse weight of 25 g; mouse lung weight of 250 mg (1% of body weight).

Table 1 shows particle size and estimated lung exposure level as a function of compound A concentration.

TABLE 1

| % Compound A | MMAD (GSD)^{a} | Estimated lung conc. (μg/g) |
|---|---|---|
| 4 | 1.35 (1.98) | 75 |
| 0.4 | 0.83 (1.87) | 11 |
| 0.04 | 0.69 (1.74) | 1.4 |
| 0.004 | 0.64 (2.35) | 0.1 |

^{a}MMAD = mass median aerodynamic diameter; GSD = geometric standard deviation

### Example 2

### Inhalation Exposure

A series of compositions for pulmonary delivery were prepared by making a 20% suspension of compound A in a 5% tyloxapol solution in water. Particle size reduction was achieved using a colloid mill (magicLAB®, MK module, for 6 hours @ 12,000 rpm, while maintaining a temperature below 50° C.). Samples at 4% w/v compound A were prepared by dilution with DI water.

Low (0.02 mg/L), mid (0.07 mg/L), and high (0.20 mg/L) target aerosol concentrations 0.3% Compound A/0.075% tyloxapol, 0.6% Compound A/0.15% tyloxapol, and 1.5% Compound A/0.375% tyloxapol suspensions were used. These low, mid, and high target formulations were prepared as follows:

4% compound A and 1% tyloxapol were removed from refrigeration and approximately 2.25, 4.5, and 12 mL were transferred into clean 50 mL graduated plastic tubes, labeled 0.3% A/0.075% tyloxapol, 0.6% A/0.15% tyloxapol, and 1.5% A/0.375% tyloxapol suspensions and allowed to warm to room temperature. Each was diluted to 20 mL with ultra pure water and vortexed until completely mixed. Each formulation was made the morning of exposure to alleviate any storing/stability issues.

### Animal Model to Test Exposure

Eighty-seven (87) male BALB/c mice (8 weeks of age on arrival and 18.6-24.6 g on exposure day) were purchased from Charles River Laboratories (Raleigh, N.C.). The three animals with the lowest body weights and the three with the highest body weights were excluded from the study on the day of randomization. The remaining eighty-one (81) animals were assigned to the study. Temperatures and relative humidity (RH) in animal housing areas were within the target range specified in the protocol.

### Inhalation Exposure

Animals were exposed to one of three concentrations of Compound A aerosols for a target of 30 min.

The actual concentrations of test article in the exposure atmospheres were determined by chemical analysis of timed filter samples collected directly from a nose-only exposure port during exposure. In addition, one impactor sample was obtained to verify particle size distribution (PSD), which is characterized by mass median aerodynamic diameter (MMAD) and geometric standard deviation (GSD). The filters and impactor stages were analyzed chemically by LRRI and are described below. Table 2 shows concentrations in the lung samples, BALF, and plasma:

TABLE 2

| Nominal Aerosol | Time point | Compound A concentration | | |
|---|---|---|---|---|
| Conc. (mg/L) | (h) | Lung (ug/g) | Plasma (ng/mL) | BALF (ng/mL) |
| 0.02 | 0.083 | 20.9 ± 13.2 | 2.16 (⅔) | 896 ± 41.7 |
| | 0.25 | 10.1 ± 1.39 | <LLOQ | 1190 ± 35.4 |
| | 0.5 | 11.7 ± 8.05 | 1.98 (⅔) | 457 ± 219 |
| | 1 | 11.8 ± 7.26 | 1.19 (⅓) | 831 ± 288 |
| | 2 | 9.55 ± 0.84 | <LLOQ | 813 ± 117 |
| | 4 | 10.5 ± 3.25 | <LLOQ | 541 ± 409 |
| | 8 | 5.66 ± 3.39 | <LLOQ | 286 ± 156 |
| | 12 | 8.67 ± 2.31 | <LLOQ | 296 ± 19.1 |
| | 24 | 3.39 ± 2.54 | <LLOQ | 223 ± 91.2 |
| 0.07 | 0.083 | 20.6 ± 10.1 | 5.43 (⅓) | 2140 ± 1340 |
| | 0.25 | 16.5 ± 2.25 | 1.03 (⅓) | 1190 ± 270 |
| | 0.5 | 16.6 ± 0.60 | 2.45 (⅓) | 1350 ± 559 |
| | 1 | 17.2 ± 2.43 | <LLOQ | 1250 ± 359 |
| | 2 | 22.7 ± 5.09 | <LLOQ | 930 ± 818 |
| | 4 | 17.0 ± 1.62 | <LLOQ | 1310 ± 1010 |
| | 8 | 15.9 ± 3.31 | <LLOQ | 800 ± 225 |
| | 12 | 16.8 ± 12.5 | 2.05 (⅓) | 569 ± 142 |
| | 24 | 10.8 ± 0.66 | <LLOQ | 512 ± 128 |
| 0.2 | 0.083 | 41.0 ± 10.3 | 1.34 (⅔) | 3150 ± 110 |
| | 0.25 | 39.2 ± 12.3 | 1.43 (⅓) | 5670 ± 2550 |
| | 0.5 | 67.2 ± 39.1 | 1.15 (⅓) | 2220 ± 1910 |
| | 1 | 37.7 ± 8.07 | 1.62 (⅔) | 2570 ± 2030 |
| | 2 | 49.3 ± 15.4 | 1.21 (⅓) | 3190 ± 2580 |
| | 4 | 44.6 ± 2.40 | 1.21 (⅔) | 3350 ± 687 |
| | 8 | 44.0 ± 12.0 | 2.19 (⅓) | 1370 ± 185 |
| | 12 | 40.8 ± 6.29 | 2.08 (⅔) | 1050 ± 799 |
| | 24 | 26.8 ± 2.63 | <LLOQ | 694 ± 56.0 |

Data are shown in mean ± standard deviation.

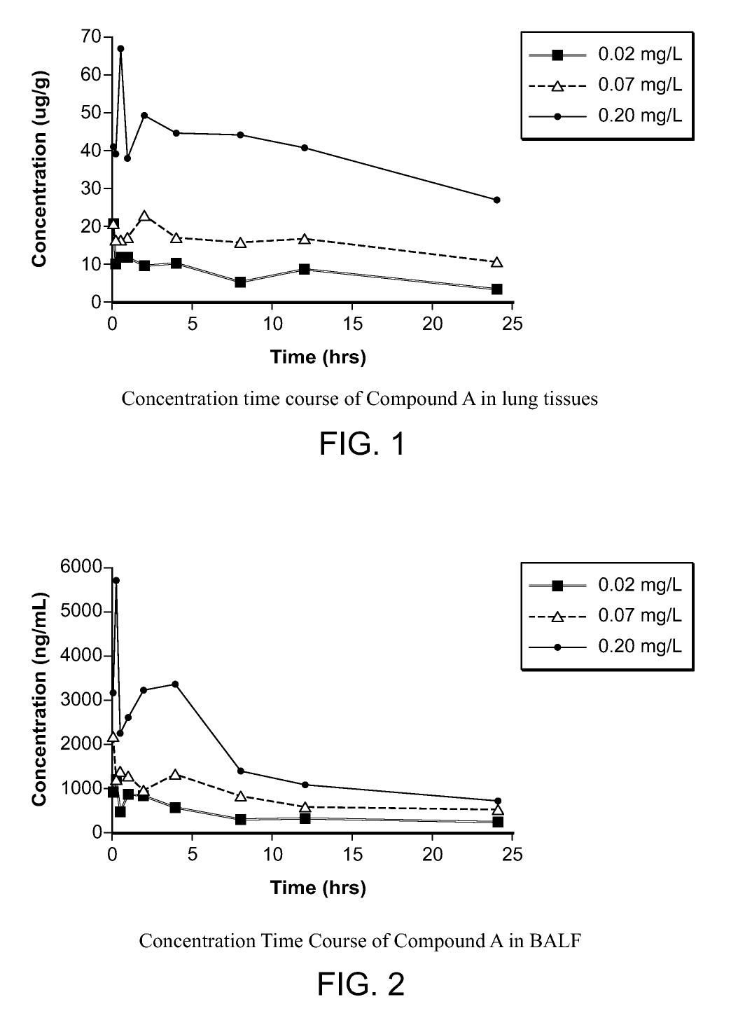

The time course of compound A concentration in the lung and BALF are depicted in FIGS. 1 and 2. The plasma data was not graphed because these levels were generally below the lower limit of quantitation (<LLOQ). As shown in FIGS. 1 and 2, the time course of compound A in both lung and BALF is dose dependent, with higher concentrations measured for the mice exposed to higher doses.

Maximum concentrations in the lung were observed at 0.08 hours (20.9 μg/g) for Group A, 2.00 hours (22.7 μg) for Group B, and 0.50 hours (67.2 μg) for Group C. Maximum concentrations in the BALF were observed at 0.25 hours (1190 ng/mL) for Group A, 0.08 hours (2140 ng/mL) for Group B, and 0.25 hours (5670 ng/mL) for Group C, as shown below in tables 3 and 4:

TABLE 3
Parameters in Mouse Lung

| | PK Parameters in Mouse Lung | | | | |
|---|---|---|---|---|---|
| Aerosol Conc. | T_{half} | Tₘₐₓ | Cₘₐₓ | AUC₍₀₋₂₄₎ | AUC_{inf} |
| (mg/L) | hrs | hrs | ug/g | hrs*ug/g | hrs*ug/g |
| 0.02 | 14.5^{#} | 0.08 | 20.9 | 176 | 247 |
| 0.07 | 24.6^{#} | 2.00 | 22.7 | 373 | 757 |
| 0.2 | 21.7 | 0.50 | 67.2 | 938 | 1777 |

^{#}Regression Coefficient is less than 0.9

TABLE 4
PK Parameters in Mouse BALF

| | PK Parameters in Mouse BALF | | | | |
|---|---|---|---|---|---|
| Aerosol Conc. | T_{half} | Tₘₐₓ | Cₘₐₓ | AUC₍₀₋₂₄₎ | AUC_{inf} |
| (mg/L) | hrs | hrs | ng/mL | hrs*ng/mL | hrs*ng/mL |
| 0.02 | 39.8 | 0.25 | 1190 | 8847 | 21657 |
| 0.07 | 16.7 | 0.08 | 2140 | 18108 | 30439 |
| 0.2 | 17.0 | 0.25 | 5670 | 37215 | 54283 |

Dose was estimated for the animals exposed to the three target concentrations. Parameters used are summarized in Table 5.

TABLE 5
Estimated Dose after a 30 Minute Inhalation Exposure

| | Target Aerosol Concentration | | |
|---|---|---|---|
| | 0.02 | 0.07 | 0.2 |
| Parameter | mg/L (A) | mg/L(B) | mg/L(C) |
| Mean Aerosol Concentration | 0.048 | 0.085 | 0.229 |
| (mg/L) | | | |
| Mean Exposure Body Weight (g) | 20.8 | 20.9 | 21.7 |
| Respiratory Minute Volume | 0.022 | 0.022 | 0.022 |
| (LPM) | | | |
| Exposure Duration (min) | 30 | 30 | 30 |
| Assumed Depostion Fraction | 0.10 | 0.10 | 0.10 |
| Dose (mg/kg) | 0.15 | 0.27 | 0.70 |

### Example 3

### Lung Exposure in Mice

A series of compositions for pulmonary delivery were prepared by making a 20% suspension of compound A in a 5% tyloxapol solution in water. Particle size reduction was achieved using a colloid mill (magicLAB®, MK module, for 6 hours @ 12,000 rpm, while maintaining a temperature below 50° C.). Samples at 4% compound A were prepared by dilution with DI water. A 0.4% compound A suspension in 0.1% tyloxapol solution, was made by dilution with SWFI (6.02 g of the 20% suspension was QS to 30.23 g with SWFI).

Each of the following solutions were prepared as indicated below:

0.4% compound A suspension in 0.1% tyloxapol solution: 2.07 g of a 4% compound A suspension was diluted to 20 g with DI water.

0.04% compound A suspension in 0.01% tyloxapol solution: 2.03 g of a 0.4% compound A suspension was diluted to 20 g with DI water. 0.004% compound A suspension in 0.001% tyloxapol solution: 2.05 g of a 0.04% compound A suspension was diluted to 20 g with DI water.

Compositions were delivered via a nose-only chamber (CH Technologies) to Balb/c mice by nebulizing for 30 minutes the composition with a PARI LC plus at 3 LPM with a 5 LPM dilutor (of dry air), tissues, plasma and BALF (3 mL used of PBS) collected after sacrificing immediately after nebulization, and were analyzed by HPLC-MS.

Particle size was measured by a 7 stage cascade impactor (mercer type) and each stage analyzed by HPLC, and the exposure of compound A in the mice is indicated in Table 6:

TABLE 6

| Formula- | | | | |
|---|---|---|---|---|
| tion | | Compound A concentration | | |
| Conc. | Animal # | Lung (μg/g) | Plasma (ng/mL) | BALF (ng/mL) |
| 0.004% | 3A | 0.12 | <LLOQ | 10.0 |
| | 3B | 0.10 | <LLOQ | 7.84 |
| | 3C | 0.10 | <LLOQ | 7.82 |
| | 3D | 0.13 | <LLOQ | 4.99 |
| | 3E | 0.14 | <LLOQ | 83.6 |
| | Mean | 0.12 ± 0.02 | <LLOQ | 22.9 ± 34.0 |
| 0.04% | 2A | 2.3 | <LLOQ | 62.6 |
| | 2B | 1.0 | <LLOQ | 93 |
| | 2C | 1.5 | <LLOQ | 92.1 |
| | 2D | 1.4 | <LLOQ | 102 |
| | 2E | 1.3 | 6.37 | 8.19 |
| | Mean | 1.48 ± 0.49 | | 71.6 ± 38.4 |
| 0.40% | 1A | 8.2 | 2.51 | 1370 |
| | 1B | 32.1(?) | 3.98 | 5240 |
| | 1C | 6.1 | 8.46 | 1200 |
| | 1D | 6.5 | 6.73 | 804 |
| | 1E | 5.9 | 4.1 | 737 |
| | Mean | 6.7 ± 1.1 | 5.16 ± 2.39 | 1870 ± 1902 |

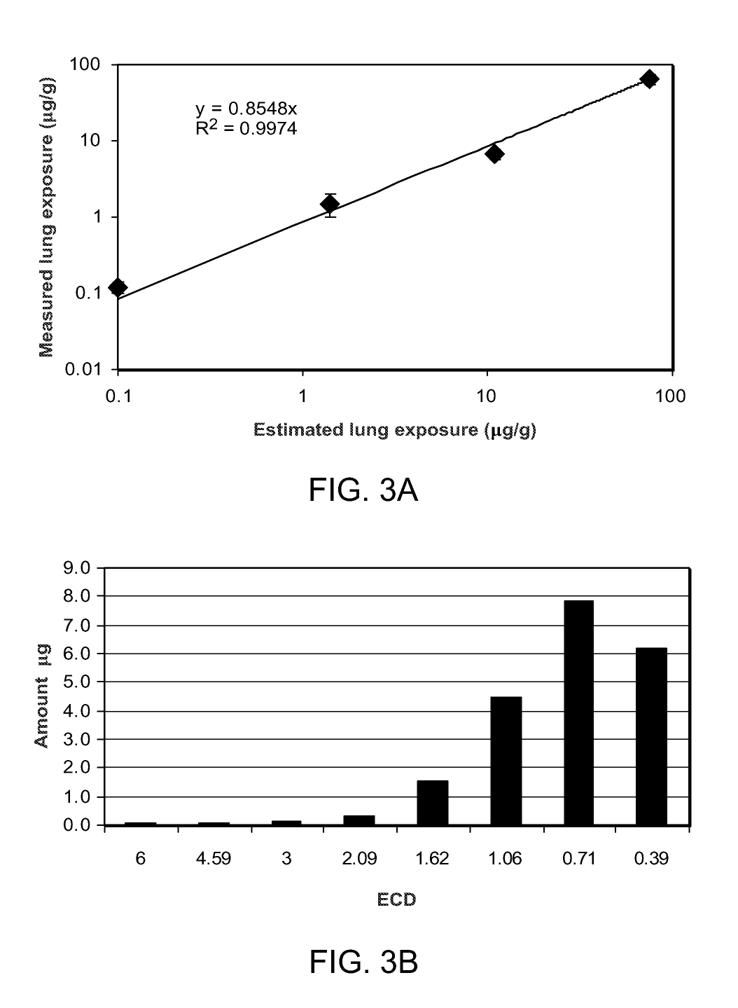

Particle size of aerosolized compositions of ˜1 μm with a GSD 1.8 μm was obtained for all compositions. An in-vivo vs in-vitro lung exposure graph was obtained by estimating the lung deposition based on exposure time, particle size, and reported lung function data of balb/c mice, and shown in FIGS. 3A and B.

### Example 4

### Mouse Airway Resistance

A series of compositions for pulmonary delivery were prepared by first preparing a 20% suspension of compound A in a 5% tyloxapol solution in water. Particle size reduction was achieved using a colloid mill (magicLAB®, MK module, for 6 hours @ 12,000 rpm, while maintaining a temperature below 50° C.). Samples at 4% compound A were prepared by dilution with DI water. A 0.4% compound A suspension in 0.1% tyloxapol solution was made by dilution with SWFI (6.02 g of the 20% suspension was QS to 30.23 g with SWFI) and the following suspensions were also prepared:
1% compound A suspension in 0.01% tyloxapol solution: 2.52 g of a 0.4% compound A suspension was diluted to 100 g with SWFI water.
0.01% compound A suspension in 0.01% tyloxapol solution: 10 g of a 0.1% compound A suspension was diluted to 100 g with SWFI water.
0.001% compound A suspension in 0.01% tyloxapol solution 3: 10 g of a 0.1% compound A suspension was diluted to 100 g with SWFI water.

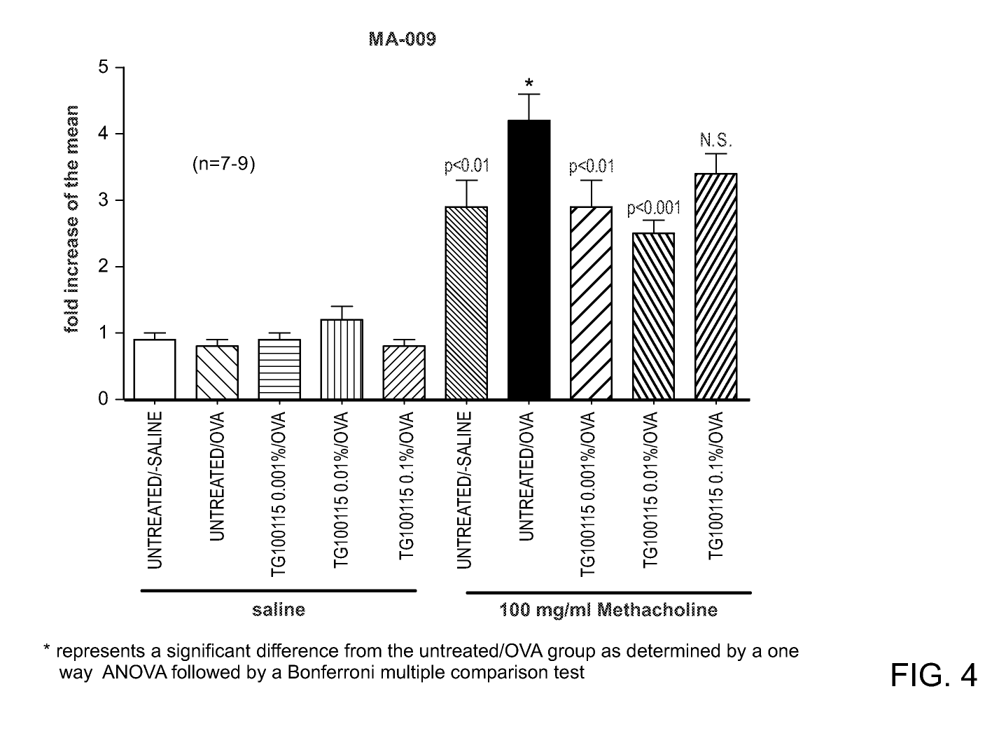

Aerosolized compound A formulations on delivery to ovalbumin (OVA) challenged mice showed reduced airway resistance, as depicted in FIG. 4. Airway resistance in the model is induced via a methacholine challenge. Airway resistance is both a diagnostic and a hallmark of respiratory diseases such as asthma and COPD and the reduction of airway resistance is considered a beneficial outcome.

### Example 5

36 mg of compound A was dissolved with the aid of 0.5 N HCl (QS to 1 g) under mixing and heat. 200 μL of this solution was mixed using a sonicator bath, if necessary with a separately prepared solution containing 1.8 mg of a 0.02% tyloxapol/2% PVP 90F and 34 μL of a 2 N NaOH, to give a final concentration of ˜3.3 mg/mL of compound A at a pH of ˜3. This solution was nebulized using a Pari LC plus- and particle size measurement was performed using a 7 stage cascade impactor resulting a in MMAD of ˜1 μm and a GSD of 2.1 (solution will be stable for more than an hour)

### Example 6

52 mg of compound A was dissolved with the aid of 0.25 N HCl (QS to 1 g) under mixing and heat. 200 μL of the above solution was mixed using a sonicator bath, if necessary with a solution containing 1.8 mg of a 0.02% tyloxapol/2% PVP 90F and 10 μL of a 2 N NaOH, to give a final concentration of ˜5 mg/mL of compound A at a pH of ˜3. This solution will start precipitating ˜20 min.

### Example 7

52 mg of compound A was dissolved with the aid of 25 μL of DMSO and 0.25N HCl (QS to 1 g) under mixing and heat. 150 μL of the above solution was mixed using a sonicator bath, if necessary with a solution containing 1.9 mg of a 0.02% tyloxapol/2% PVP 90F and 7.6 μL of a 2 N NaOH, to give a final concentration of ˜3.7 mg/mL of compound A at a pH of ˜3

The diluted solution is made prior to nebulization, while the concentrated acidic formulation is made in advance. If some precipitate is observed in the concentrated stock solution-heating to 40-50° C. usually dissolves the compound.

### Example 8

A series of dry powder formulations of compound A were manufactured by lyophilizing suspensions of compound A at 2% in a 0.5% tyloxapol solution in water (from example 4), with additional excipients with the following compositions 1) Compound A: tyloxapol 4:1 w/w, 2) Compound A: Dimyristoylphosphatydyl choline (DMPC): m-Hetastarch: tyloxapol 4:4:4:1 w/w/w/w. 3) Compound A: Dimyristoylphosphatydyl choline (DMPC): tyloxapol 4:4:1 w/w/w/w, 4) Compound A: Hydrogenated phosphatidylcholine from Soy (PL90H): m-Hetastarch: tyloxapol 4:4:4:1 w/w/w/w, 5) Compound A: Hydrogenated phosphatidylcholine from Soy (PL90H): tyloxapol 4:4::1 w/w/w/w.

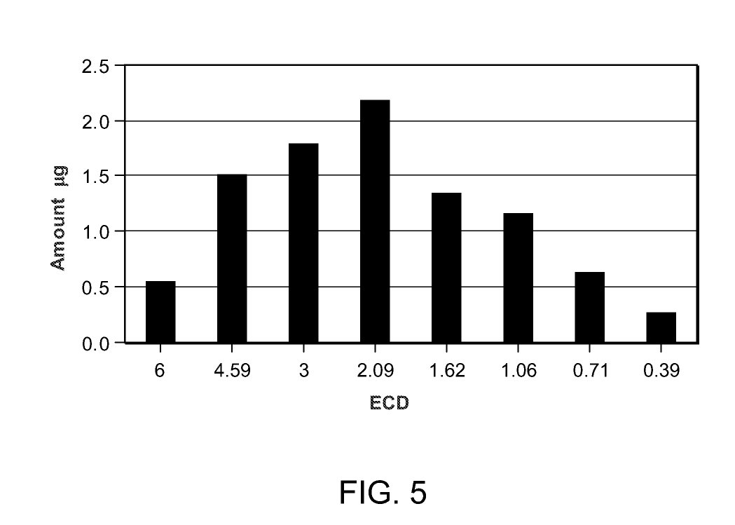

The dry powders were aerosolized with an active dry powder insufflator (PennCentury) equipped with a small spacer to select the majority of the fine particles. Particle size distribution was measured using a 7 stage cascade impactor (Intox) at 1 LPM. All powders showed to exhibit a particle size of ˜3 μm with a geometric standard deviation of ˜2.2 μm. FIG. 5 depicts the particle size distribution (effective cut diameter of an aerosolized dry powder with a composition of Compound A: dimyristoylphosphatydyl choline (DMPC): m-Hetastarch: tyloxapol 4:4:4:1 w/w/w/w.

### REFERENCES

All publications and patents mentioned herein, including those items listed below, are hereby incorporated by reference in their entirety as if each individual publication or patent was specifically and individually incorporated by reference. In case of conflict, the present application, including any definitions herein, will control.

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

1. A method for targeting delivery of a pharmaceutical kinase inhibitor composition to the respiratory tract of a patient in need thereof, comprising:
administering to the patient a therapeutically effective amount of pharmaceutically acceptable composition suitable for direct delivery to the respiratory tract comprising:
(a) from about 0.00001 to about 10% w/v of a kinase inhibiting agent selected from: 6,7-bis(3-hydroxyphenyl)-pteridine-2,4-diamine or pharmaceutically acceptable salts thereof; and
(b) from about 0.00001 to about 10% w/v of a surfactant,
wherein the administration results in the drug being predominantly delivered to a mucosal surface of the respiratory tract of the patient and results in a minimal plasma concentration of the kinase inhibiting agent in the patient.

2. The method of claim 1, wherein the pharmaceutically acceptable composition further comprises an aqueous based solvent.

3. The method of claim 1, wherein the plasma concentration of the agent in the patient is less than about 10 ng/mL within about two hours after administration.

4. The method of claim 1, wherein the plasma concentration of the agent is less than about 2 ng/mL.

5. The method of claim 1, wherein the surfactant is selected from one or more of: tyloxapol, poloxamer 188, poloxamer 407, Tween™ 80, phosphatidylcholine, phosphatides, or phosphatidyl glycerols.

6. The method of claim 1, wherein the surfactant is tyloxapol.

7. A composition suitable for administrating by inhalation or nasally, comprising:
a) an active agent represented formula I: 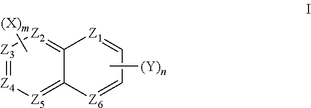
wherein:
each of Z₂ and Z₄ is C, each of Z₁, Z₃, Z₅, and Z₆ is N;
each X is NH₂;
each Y is independently selected from a group consisting of —OR^{d}, —NR^{d}₂, —SR^{d}, and —OPO₃H₂, wherein Rd is selected from a group consisting of H, lower alkyl, aryl, and —(CH₂)₂NH(CH₂CH₃); or
each Y is independently selected from a group consisting of alkyl, substituted alkyl, aryl, substituted aryl, and halogen, wherein said substituents are selected from a group consisting of halogen, —OR^{e}, —NR₂—SR^{e}, and —P(O)(OH)₂, wherein R^{e} is selected from a group consisting of —H, lower alkyl, and aryl; or
each Y is independently selected from a group consisting of CH₂glycinyl, CH₂NHethoxy, CH₂NHCH₂alkyl, CH₂NHCH₂t-Bu, CH₂NHCH₂aryl, and CH₂NHCH₂substituted aryl; or
when n is 2, each Y is taken together to form a fused aromatic ring system; and m and n are each independently 1 to 4, or pharmaceutically acceptable salts thereof; and
b) a surfactant.

8. The composition of claim 7, comprising:
a) about 0.00001% w/v to about 20% w/v of 6,7-bis(3-hydroxyphenyl)-pteridine-2,4-diamine or pharmaceutically acceptable salts thereof;
b) about 0.00001% w/v to about 6% w/v of a surfactant; and
c) water sufficient to make 100% w/v.

9. The composition of claim 7, wherein the surfactant is selected from one or more of: tyloxapol, poloxamer 188, poloxamer 407, Tween™ 80, phosphatidylcholine, phosphatides, and phosphatidyl glycerols.

10. The composition of claim 7, wherein the surfactant is tyloxapol.

11. The composition of claim 7, wherein the composition is in the form of a nebulized aerosol or a powder.

12. The composition of claim 8, wherein the composition, when administered to a patient as a nebulized aerosol or dry powder, results in a lung tissue concentration in the patient of at least about 10 ng/g of 6,7-bis(3-hydroxyphenyl)-pteridine-2,4-diamine or pharmaceutically acceptable salts thereof about 15 minutes after administration.

13. The composition of claim 8, wherein the composition, when administered to a patient as a nebulized aerosol or dry powder, results in a plasma concentration in the patient of less than about 5 ng/mL about 15 minutes after administration.

14. A method of treating asthma or COPD of a patient in need thereof comprising:
administering by inhalation to the patient a composition comprising a therapeutically effective amount of the active agent 6,7-bis(3-hydroxyphenyl)-pteridine-2,4-diamine or pharmaceutically acceptable salts thereof and a surfactant,
thereby delivering to the mucus membranes of the lungs of the patient a pharmaceutically effective amount of the active agent.

15. The method of claim 14 wherein the method of treating COPD is a method of treating emphysema.

16. The method of claim 14, wherein the method of treating COPD is a method of treating chronic bronchitis.

17. The method of claim 14, wherein the surfactant is selected from one or more of: tyloxapol, poloxamer 188, poloxamer 407, Tween™ 80, phosphatidylcholine, phosphatides, and phophatidyl glycerols.

18. The method of claim 14, wherein the surfactant is tyloxapol.

19. A kit for treating a respiratory disease, the kit comprising (a) a composition comprising 6,7-bis(3-hydroxyphenyl)-pteridine-2,4-diamine or pharmaceutically acceptable salts thereof, and a non-ionic surfactant, in a sealed container, and (b) a label indicating administration by inhalation or nasally.

20. The kit of claim 19, wherein the composition comprises 0.05 mg to 40 mg of 6,7-bis(3-hydroxyphenyl)-pteridine-2,4-diamine or pharmaceutically acceptable salts thereof.
